(19) United States
(12) Plant Patent Application Publication
(10) Pub. No.: US 2007/0266466 P1
(43) Pub. Date: Nov. 15, 2007

(54) HOMOZYGOUS MALE ASPARAGUS PLANT NAMED 'NJ22-34'

(75) Inventors: Stephen A. Garrison, Pittsgrove, NJ (US); Chee-kok Chin, Holmdel, NJ (US); John J. Kinelski, Princeton, NJ (US)

Correspondence Address: James A. Lucas;Driggs, Hogg & Fry CO.,L.P.A., 38500 Chardon Road, Willoughby Hills, OH, 44094, US

(73) Assignee: The State University, New Brunswick, NJ (US)

(21) Appl. No.: 11/431,329

(22) Filed: May 10, 2006

(51) Int. Cl. A01H 5/00

(52) U.S. Cl. PLT/260

(57) ABSTRACT

A new and distinct asparagus (Asparagus officinalis Linn.) plant denoted ‘NJ22-34’ which is homozygous for male sex gene was developed through extensive breeding and selection. Asparagus hybrid ‘NJ22-34’ has many desirable traits including homogenous in male genes, good resistance to rust (Puccinia asparagi) and good field tolerance to asparagus root rot (Fusarium oxysporum) and asparagus crown rot (Fusarium moniliforme). Because ‘NJ22-34’ is a homozygous-male plant all its Fl progenies will be male plants. This invention relates to the plant and plant parts of ‘NJ22-34’. The invention further relates to hybrid asparagus seeds and plants produced by crossing asparagus plant ‘NJ22-34’ with another asparagus plant.

### CROSS REFERENCE TO RELATED APPLICATIONS

This application is related to co-pending plant patent application Ser. No. 11/431,187 entitled GENOTYPE OF ASPARAGUS PLANT NJ 44P which is owned by the assignee of the present invention and is filed currently herewith.

### LATIN NAME OF THE GENUS AND SPECIES

The Latin name is Asparagus officinalis.

### VARIETY DENOMINATION

The varietal denomination is ‘NJ22-34’.

### BACKGROUND OF THE INVENTION

For an asparagus plant to be commercially viable and profitable, good yield is essential. It is well known that asparagus is susceptible to a number of diseases. Among the most devastating are rust caused by Puccinia asparagi d.c., (Kahn et al. 1952), crown rot caused by Fusarium oxysporum and root rot caused by f. Proliferatum (syn=f. Moniliforme) (Johnston et al., 1979; Guerrero et al., 1999). Of course, the presence of these diseases aversely impacts the yields and therefore the profitability of the product. Accordingly, resistance to these diseases is essential to commercial success.

### BRIEF SUMMARY OF THE INVENTION

This invention relates to a new and distinct homozygous-male asparagus plant designated as ‘NJ22-34’. Plant ‘NJ22-34’ possesses several desirable traits including vigorous growth habit, resistance to rust (Puccinia asparagi), and good field tolerance to root and crown rot (Fusarium oxysporum) and (Fusarium mondiforme), respectively, as compared to its progenies.

### BRIEF SUMMARY OF THE DRAWINGS

The color notations in our Data Summary have been selected by us from observations as compared with the Munsell Limit Color Cascade, Munsell Color. It should be mentioned that foliage color of asparagus could be affected by many factors including plant nutrition, temperature, humidity and the density of growth. Therefore, the color characteristics of this new variety should be determined with reference to the observations described herein, rather than from this illustration alone. Furthermore, the coloration is not considered to be a distinguishing feature of this new variety.

FIG. 1 shows in color a typical planting of ‘NJ22-34’ as it appears in a field under normal conditions.

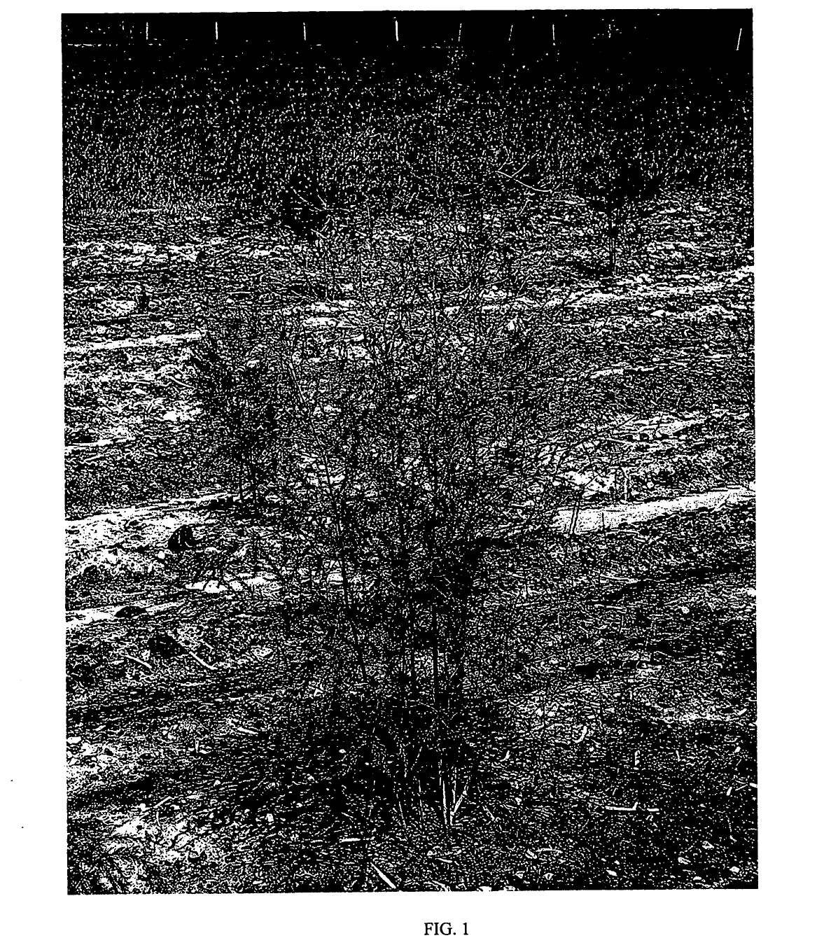

### DETAILED DESCRIPTION OF THE INVENTION

An extensive program of asparagus plant development, carried out at a cultivated area in the vicinity of Bridgeton, N.J. and New Brunswick, N.J. has resulted in the development of the asparagus hybrid plant ‘NJ22-34’ with many desirable traits. ‘NJ22-34’ was produced by self-fertilization of an andromonoecious male plant known as Asparagus Plant No. 22 (U.S. Plant Pat. No. 4,999). Asexual reproduction of ‘NJ22-34’ has been conducted via tissue culture in a laboratory in New Brunswick, N.J.

Asparagus (Asparagus officinalis Linn.) is a dioecious species with individual plants being either male or female in sex. In addition to differences in morphology “cultivars” may also differ in local adaptation, yield, disease resistance, and longevity. Desirable cultivars are developed by crossing of elite male and female parents. Both male and female parents transmit traits such as disease resistance, yield, and spear morphology to their progenies. ‘NJ22-34’ is produced by self-fertilization of an andromonoecious plant ‘NJ22’. The asparagus plant ‘NJ22-34’ is a homozygous male. All of the Fl progenies produced by crossing any female with ‘NJ22-34’ will be males.

Good yield is essential for profitability. Yield of asparagus may vary significantly among genotypes. In tests carried out in different locations, hybrids of crosses of different females and ‘NJ22-34’ produced higher yield than hybrids of crosses of different females and previously patented asparagus plant 22-8 (U.S. Plant Pat. No. 5,549, Ellison).

The following table shows that the progenies of ‘NJ22-34’ out yield the progenies of NJ 22-8, which is the parent of Jersey Giant (U.S. Plant. Pat. No. 5,551) and Jersey Knight (U.S. Plant Pat. No. 6,624), two highly popular asparagus varieties cultivated around world today.

TABLE 1

| Crosses | Name | Yield, LB/A |
|---|---|---|
| NJ56 × NJ22-34 | NJ854 (US PP16140) | 4881 |
| G27 × NJ22-34 | Not named | 4424 |
| NJ277C × NJ22-34 | Not named | 4215 |
| G27 × NJ22-8 | Jersey Gem (US PP6970) | 3673 |
| NJ362M × NJ22-34 | Not named | 3645 |
| NJ56 × NJ22-8 | Jersey Giant | 3595 |
| NJ277C × NJ22-8 | Jersey Knight | 3278 |
| NJ362M × NJ22-8 | Jersey General (US PP6965) | 3155 |

Asparagus can be clonally propagated by crown division. Crown is the structure where shoots and roots join together. Division or separation at the crown area will allow the propagation of asparagus. It has been observed that ‘NJ22-34’ plants propagated by crown division have similar morphological appearance and possess the same desirable characteristics as the original ‘NJ22-34’.

Asparagus can also be clonally propagated by tissue culture. An asparagus shoot tip or meristem when cultured on appropriate nutrient medium and appropriate conditions can grow, develop, and regenerated into a plant. Also, asparagus plant part such as a spear segment when cultured on appropriate nutrient medium and appropriate conditions can grow, develop, and regenerate into asparagus plant. Such plant can be efficiently divided and multiplied in appropriate nutrient medium. The ‘NJ22-34’ plant propagated by various tissue culture techniques possesses and retain the same desirable characteristics as the original ‘NJ22-34’ plant.

This invention also includes hybrids produced by crossing a female asparagus plant with ‘NJ22-34’. When crossed with different female plants, asparagus ‘NJ22-34’ produced only male hybrids. ‘NJ22-34’ can transmit its desirable traits including vigorous growth habit, resistance to rust (Puccinia asparagi) good field tolerance to root and crown rot (Fusarium oxysporum) and (Fusarium mondiforme) to its progenies.

Morphological data has been accumulated that distinguishes asparagus plant NJ22-34 from other asparagus varieties that developed internally, as well as asparagus plants that are known and available commercially in the markets.

### BOTANICAL DESCRIPTION

The data (averages from 3 clones) are assembled in the following table:

### ASPARAGUS PLANT: ‘NJ22-34’

TABLE 2
Stalk data:
Number of nodes below first branch: 29.3.
Distance from crown to first branch: 55.8 cm.
Number of branches: 53.
Distance between first and last branch: 118.96 cm.
Internode length between branches: 2.24 cm.
Number of cladophyll nodes beyond last branch: 31.67.
Length beyond last branch: 13.33 cm.
Largest stalk diameter: 15 mm.
Mean diameter of three largest stalks: 13.33 mm.
Number of stalks: 38.
Mature stalks color, bloom removed: Color No. 22-11.
Typical and observed branch color: Color No. 19-11.
Flower
Typical number per cluster: 2.
Tepal - 6 per flower.
Apex outer surface margin Color No. 23-6.
Apex inner surface margin Color No. 25-5.
Apex outer surface middle (vertical) area Color No. 47-5.
Apex inner surface middle (vertical) area Color No. 23-5.
Base outer surface margin Color No. 23-8.
Base inner surface margin Color No. 25-3.
Base outer surface middle (vertical) area Color No. 47-8.
Base inner surface middle (vertical) area Color No. 23-8.
Shape - elliptic, average 7.8 mm long. 3.2 mm wide at mid point,
slightly wider towards apex, fused in lower third into a tubular
corolla, imbricate in bud; apex obtuse; base cunneate; margin entire.
Flower length: 7.8 mm.
Flower width at midpoint: 3.2 mm.
Pedicel: filiform: length - 16 mm; no bract.
Cladophyll
Average number per node: 7.8.
Shape: linear, filiform, needle-like; apex acute; base cuneate,
margin - entire;
Color - 20-11.
Average length of cladophylls on first node: 25 mm.
Average width of cladophylls on first node: 0.8 mm.
Leaves
Main stem leaves scale-like, triangular, average 13 mm long, 12 mm
wide at base, membranous: apex acuminate; base truncate; margin
hyaline; Color - 28-9 abaxial; 28:8 adaxial.
Terminal branch leaves scale-like, triangular, average 5 mm long,
3 mm wide at base, membranous; apex acuminate; base truncate; margin
erose, hyaline. Color: 28-8 abaxial; 28-7 adaxial.
Reproductive organs
Stamens - 6 in number, each stamen fused to middle of inner side
of a tepal.
Filaments - filiform, straight, slightly wider at base 4.5 mm long;
Anthers elliptic, longitudinally dehiscent, introse to slightly
latorose, base sagittate, apex obtuse with small acuminate tip.

1. A new and distinct asparagus plant ‘NJ22-34’, which is homozygous for male sex gene as herein shown and described.
